(19) United States
(12) Patent Application Publication
(10) Pub. No.: US 2009/0280029 A1
(43) Pub. Date: Nov. 12, 2009

(54) High Throughput Materials-Processing System

(76) Inventors: Youshu Kang, Shanghai (CN); Guilin Wang, Shanghai (CN); Wenge Wang, Shanghai (CN); Wenhui Wang, New York, NY (US); Youqi Wang, Palo Alto, CA (US); Guangping Xie, Shanghai (CN); Sibiao Xu, Shanghai (CN); Xiaowen Zhu, Shanghai (CN); Xianzhong Zhao, Shanghai (CN)

Correspondence Address: PERKINS COIE LLP, POST OFFICE BOX 1208, SEATTLE, WA, 98111-1208, US

(21) Appl. No.: 11/914,216

(22) PCT Filed: May 10, 2006
(86) PCT No.: PCT/CN2006/000938

§ 371 Date: Nov. 12, 2007

Related U.S. Application Data
(60) Provisional application No. 60/680,300, filed on May 11, 2005.
(60) Provisional application No. 60/689,649, filed on Jun. 9, 2005.

(51) Int. Cl. G01N 33/00

(52) U.S. Cl. 422/63

(57) ABSTRACT

The present invention discloses a materials-processing system, which comprises an inputting subsystem, a processing apparatus coupled to the inputting subsystem and a collecting subsystem coupled to the processing apparatus. The inputting subsystem comprises three or more sample vessels, which can be connected to the processing apparatus. Since the processing system includes multiple sample vessels, which can be grouped into different groups so that each group contains two or more of the multiple sample vessels. High throughput materials transport can be realized by sequentially connecting different groups of sample vessels to the processing apparatus, thereby overcoming a limitation of the prior art that cannot continuously perform multiple batches of materials processing and improving material processing efficiency.

### FIELD

The invention relates to a high throughput materials-processing system

### BACKGROUND

A known processing system, such as the processing system disclosed in U.S. Pat. No. 6,471,392, generally comprises a processing reactor and two sample vessels connecting to the processing reactor through respective conduits. When this kind of processing system is used to process multiple batches materials, a first batch of materials is usually processed firstly, which involves transporting materials stored in the sample vessels into the processing reactor and transporting the materials after processing to a collecting vessel via an outlet of the processing reactor. Thereafter, the system is cleaned so as to avoid cross contamination of different batches of materials before a second batch of materials can be processed. This kind of processing system when used to process multiple batches of materials is obviously inefficient as reflected in the following three aspects:

Firstly, after processing each batch of materials, the next batch of materials are provided either by way of disassembling the sample vessels and replacing them with new sample vessels loaded with the next batch of materials, or by way of cleaning the sample vessels and loading the cleaned sample vessels with the next batch of materials. No matter which way is used, manual operation is needed and considerable time is consumed. Additionally, the processing system with only two material transporting passages can not deal with processing of three or more materials at one time.

Secondly, in an aspect of product collection, there are also two ways: disassembling the collecting vessel from the processing reactor and replacing them with a new collecting vessel, or removing products from the collecting vessel. Similarly, manual operation is needed and considerable time is consumed.

Thirdly, in the aspect of system cleaning, similar to processing of a batch of materials, there are also two ways: replacing the sample vessels with new ones or replacing samples in the sample vessels with materials for cleaning. If the former way is used, disassembling and installation of sample vessels are needed. If the latter way is used, the sample vessels and their transporting passages should also be cleaned. Therefore, no matter which way is chosen, manual work and time are consumed.

Furthermore, the manual operations involved in the aforementioned procedures may increase both risk of mishandling and cost.

Due to the deficiency of the current material processing system in these three aspects, this kind of processing system is not suitable for processing batches of materials in a high throughput manner. Therefore, there is a need for a high throughput processing system for continuously processing multiple batches of materials.

Moreover, since the aforementioned processing system has only two sample vessels connected to the reactor, and thus cannot handle processing of three or more materials at one time, there is also a need for a high throughput processing system to process more than two materials at one time.

### SUMMARY OF THE INVENTION

An aspect of the present invention provides a high throughput materials-processing system, which comprises an inputting subsystem, a materials-processing apparatus coupled to the inputting subsystem and a collecting subsystem coupled to the processing apparatus. The processing apparatus is used to process materials and includes a processing chamber. The collecting subsystem is used for collecting materials processed by the processing apparatus. The inputting subsystem comprises multiple sample vessels for storing material samples, and each sample vessel can be connected to the processing chamber such that the material samples stored therein can be transferred into the processing chamber.

The inputting subsystem comprises three or more sample vessels. There is no limitation as to the specific number of the sample vessels, and it may be 3, 4, 5, 6, 7, 8, 9, 10, 16, 20, 32, 40, 80, 128, etc. The sample vessel includes a receiving room for storing a material sample.

The sample vessel and the processing apparatus can be connected in various ways. In one embodiment, the sample vessel is connected to the processing apparatus through a connection device in between. In another embodiment, the sample vessel is directly connected to the processing apparatus. More details about the connection ways will be described below in conjunction with different embodiments.

As to the way that the sample vessel is connected to the processing apparatus through a connection device, there may be a fixed connection manner and a movable connection manner. Various connection devices can be used. For example, the connection device may be a device defining a transportation passage therein, such as a pipe known by the art. Additionally, the connection device may further comprise a connection element, and the sample vessel is firstly connected to the connection element, and the connection element is connected to the processing apparatus through a pipe or the like. The connection element can be any element with the connection function known by the art. In one embodiment, it can be a selective connection element defining therein two or more passages, which can be selectively connected to one or more sample vessels. For example, it can be a gate valve known by the art, such as four-way valve, six-way valve and etc, and it also can be a docking element. As known by those skilled in the art, the connection device has a plurality of different embodiments. More details about the connection devices will be described in conjunction with different embodiments hereafter.

As to the fixed connection manner, the sample vessels, the materials-processing apparatus and the connection device therebetween are not movable relative to each other when they are in assembly.

An embodiment of a fixed connection manner is illustrated in FIG. 1. Multiple sample vessels 101, 102, 103 are connected to a processing apparatus 100 through pipes 111, 112, 113 (disposed between the sample vessels 101, 102, 103 and the processing apparatus 100).

Another embodiment of a fixed connection manner is illustrated in FIG. 2. Multiple sample vessels 201, 202, 203, 204 are connected to a connect element 220 through pipes 211, 212, 213, 214 (disposed between the sample vessels 201, 202, 203, 204 and the connect element 220) and the connect element 220 is connected to the processing apparatus 200 through a pipe 221 (disposed between the connect element 220 and the processing apparatus 200). There is no limitation as to the number of the connect elements used, the number of the sample vessels connected to one connect element, or the number of the connect elements connected to the processing apparatus. For example, in one embodiment, referring to FIG. 3, a plurality of sample vessels 301, 302, 303, 304, 305, 306, 307, 308, 309 are grouped into three groups 30, 31, 32, and numbers of the sample vessels in different groups are different. However, different groups may have a same number of sample vessels in other embodiments. The sample vessels of the first group 30 are connected to a first connect element 321 through pipes 311, 312, 313, 314 (disposed between the sample vessels and the connect element), and the first connect element is connected to the processing apparatus 300 through pipe 331 (disposed between the connect element and the processing apparatus). The sample vessels 305, 306, 307, 308, 309 of the other groups 31, 32 are correspondingly connected to respective second and third connect elements 322, 323 through pipes 315, 316, 317, 318, 319 (disposed between the sample vessel and the connect element). The second and third connect elements 322, 323 are connected to a fourth connect element 324, and the fourth connect element 324 is connected to the processing apparatus 300 through pipe 334 (disposed between the connect element and the processing apparatus)

Further, the above embodiments of the fixed connection manner disclosed in FIGS. 1, 2 and 3 can be used in combination with each other. These combinations can be simply established by those skilled in the art and therefore are not again illustrated with reference to figures.

As to the movable connection manner, at least one of the sample vessels, the connect element and the processing apparatus is movable relative to another, and the connection between the sample vessel and the processing apparatus is realized by the movement. For example, in one embodiment, the connection between the sample vessel and the processing apparatus is realized by the movement of the connect element. The sample vessels or the processing apparatus may additionally or alternatively be movable, which depends on design requirement. The moveable sample vessel, connect element, or processing apparatus can be driven by any known driving methods, such as, by a pneumatic cylinder, electric motor or plunger driver, etc. Since the technology about driving methods is well known, no more unnecessary descriptions will be given on these driving methods. More about the movable connection manner will be given by illustrative embodiments as follows.

An embodiment, in which sample vessels and a processing apparatus are connected through a connection device in a movable connection manner, is illustrated in FIG. 4. A plurality of sample vessels 401, 402, 403, 404, 405 are provided. A connection element 410 includes a receiving room 412 (figured by the broken line) and a docking port 414. The connection element 410 is movable (the movement track of the connection element 410 is figured by the broken line in FIG. 4, and the movement manner can be any movement manners known by the art, and can be different in different embodiments). When a material stored in a selected one of the sample vessels 401, 402, 403, 404 and 405 is wanted to be transported into the processing apparatus 400, the connection element will move to a position of the selected sample vessel and is connected to the sample vessel 401, 402, 403, 404, 405 through its docking port 414. After the material stored in the sample vessel is transported into the receiving room 412, the connection element 410 will move in order to connect to an input port 416 of the processing apparatus 40 so as to transport the material into the processing apparatus 40. The movement of the connection element can be realized by a robot technology as known, that is to say, a robot which can move in a space and includes a medi-vessel may move and have the medi-vessel sequentially connected to the selected sample vessel and to the processing apparatus, such that the material stored in the selected sample vessel can be transported to the processing apparatus. In another embodiment, the sample vessels 401, 402, 403, 404, 405 move to connect to the connection element 410 by themselves, and the connection element 410 moves to connect to the processing apparatus, so as to transport the material stored in the selected sample vessel to the processing apparatus.

Another embodiment, in which sample vessels and a processing apparatus are connected through a connection device in a movable connection manner, is illustrated in FIG. 5. A plurality of sample vessels 501, 502, 503, 504, 505 are provided. A connection element 510 has two ends, one of which is connected to a processing apparatus 500 through a pipe 521, and the other of which defines a movable docking port 511 (the movement track of the connection element 510 is figured by the broken line in FIG. 5, and the movement manner can be any movement manners known by the art, and can be different in different embodiments). The docking port of the connection element 510 can move to connect to any selected sample vessel 501, 502, 503, 504, 505, so as to complete the connection between the selected sample vessel and the processing apparatus. In another embodiment, the sample vessels 501, 502, 503, 504, 505 and the connection element 510 are movable, and the connection between the sample vessel and the processing apparatus can be completed through movements and engagements of the sample vessel and the connection element.

The above embodiments about the movable connection manners as disclosed in FIGS. 4 and 5 can be used in combination with each other. These combinations can be simply established by those skilled in the art and therefore are not again illustrated with reference to figures. There will be no restriction as to the numbers of the connection elements 410, 510 used, and they may change depending on the specific situations. For example, there may be two connection elements capable of having two selected sample vessels from all sample vessels connected to the processing apparatus at one time.

Further, the embodiments of the fixed connection manner and the movable connection manner disclosed above also can be used in combination with each other. In one embodiment, referring to FIG. 6, there is a plurality of sample vessels 601, 602, 603, 604, some of which connect to a processing apparatus through a connection element in a fixed connection manner. For example, the sample vessels 601, 602 connect to a first connect element 620 through respective pipes 611, 612, and a first connection element 620 connect to the processing apparatus 600 through a pipe 621. The rest of the sample vessels connect to the processing apparatus in a movable connection manner. There is a second element 630, which has a first end connecting to the processing apparatus 600 with a pipe 631, and a movable second end defining a docking port for connecting to the sample vessel. The second connection element can be connected to any selected sample vessel 603, 604 through a movement of its second end (the movement track of the second connection element 630 is figured by the broken line in FIG. 6, and the movement manner can be any movement manner known by the art, and can be different in different embodiments.

Further, a plurality of sample vessels can be disposed on a base, which can be movable or immovable according to actual needs. Some illustrative different embodiments will be provided as follows.

In one embodiment, referring to FIG. 7, an inputting subsystem comprises a plurality of sample vessels 701, 702, 703, 703, 704, 705 arranged on a disc-shaped base 710, which can rotate around its middle-axis along a direction indicated by the arrow as shown in the figure. An end of a connection element 720 connect to the processing apparatus 700 through a pipe 722, and the other end of the connection element 720 is movable along an up-down direction and defines a docking port 724. The base 710 rotates to cause a selected sample vessel placed rightly under the movable end of the connection element 720, and then the movable end of the connection element 720 moves downwards to have its docking port 724 connected to the selected sample vessel, so as to complete the connection between the selected sample vessel and the processing apparatus 700. The shape and the movement manner of the base, the arrange manner of the sample vessels on the base and the movement manner of the connection element disclosed here are for only exemplary illustration, and they may vary in other embodiments. These elements engage with each other to achieve a final aim: completing the connection between the selected sample vessel and the processing apparatus, so as to finish the transportation of the material sample from the sample vessel to the processing apparatus. The transportation manners of the sample from the sample vessel to the processing apparatus can be any one as disclosed above or others known by the art. For example, the base may be in a rectangular shape, and a plurality of sample vessels are linearly arranged on the base. The base is linearly movable so that a selected sample vessel can be transported to a predetermined position by the linear movement of the base, such that the connection element can move downwards to connect with the selected sample vessel in the predetermined position. In another embodiment, the selected sample vessel is transported to a predetermined position by the base and moves itself to connect with the connection element.

Further, in order to achieve a fitting connection between the connection element and the selected sample vessel, the sample vessels may be mounted on the base in such a manner that the sample vessels are immovable relative to the base along a direction, but are movable to some extent in other directions. For example, referring to FIG. 8, a plurality of sample vessels 801, 802, 803, 804, 805 are loosely received in a plurality of receiving rooms 811, 812, 813, 814, 815 defined in a base 810, respectively, and there are spaces 816 between the sample vessels and the receiving rooms. The sample vessels are immovable in up-down directions, but are slightly movable along other directions. For example, the sample vessels may move in horizontal directions to adjust their positions, so as to achieve fitting connections with the connection element.

Further, a sealing element will be provided to ensure a sealing effect of the connection between the sample vessel and the connection element, so as to avoid material leak. The sealing element may be made from elastic material, “O” shape rings or any other sealing element known by the art. Another method to enhance the sealing effect is to design connecting parts of the sample vessel and the connection element in shapes or forms benefiting sealing. For example, the connecting parts of the sample vessel and the connection element may be configured to linearly contact with each other. In one embodiment, referring to FIG. 9, the sample vessel 900 has a outlet 901 configured in a round shape, and the connection element has an engaging port 911 configured in a cone-like shape for engaging with the round outlet 901 of the sample vessel. In assembly, a receiving room 902 in the sample vessel 900 communicates with a passageway 912 in the connection element 910. The outlet 901 and the engaging port 911 are brought into contact with each other at two linear contact portions, thus a better sealing effect can be achieved. In other embodiments, maybe the port of the sample vessel is cone-shaped and the engaging port of the connection element is in round shape; or maybe both the port of the sample vessel and the engaging port of the connection element are cone-shaped or in round shape. The sample vessel and the connection element alternatively may be configured into other shapes to achieve linear contact therebetween.

The sample vessel for storing samples can be any container, collector or any device with a function for storing known by the art. The present invention also discloses a syringe type sample vessel. Referring to FIG. 10, a sample vessel 150 comprises a body 152 defining a receiving room 153 for storing samples. The body 152 has an end defining a inlet 154 communicating with the receiving room 153 and another end engaging with a compress element 155. The compress element can enter the receiving room 153 to push a sample stored in the receiving room 153 out of the receiving room 153 through the inlet 154. Moreover, a sealing element can be provided between the compress element and the receiving room, so as to prevent leakiness while the compress element is pushed into the receiving room. The sealing element can be “O” shape ring, etc, and the compress element can be driven by motor, cylinder, piston, etc.

Further, when the sample vessel is connected to the processing apparatus, the sample can be transported from the sample vessel to the processing apparatus in various transportation manners. For example, the sample may enter the processing apparatus by itself as a result of its own weight. A power device may be used to provide a power to push the sample into the processing apparatus. The power device may be mounted in various manners. For example, it can be connected to the sample vessel through a pipe, or connected to the movable connection element, which is connected to the processing apparatus. Whatever, the only requirement is that a power can be provided to move the material sample stored in the sample vessel. The power device can be any power device known by the art, such as, pump, pressurize device or piston, etc.

In another embodiment, referring to FIG. 11, a plurality of sample vessels 170 are connected to a connection element 174 through the pipes 172 respectively, and the connection element 174 is connected to the processing apparatus 180 through a pipe 176 (in other embodiments, the connection manner can be a movable connection manner as disclosed above). A pressurize device 190, which is used as a power device, comprises a gas source 192, a valve 194 and a distribution device 196. All the sample vessels are connected to the gas source through the distribution device. For example, the sample vessel 170 is connected to the gas source through a pipe 198 of the distribution device 196, such that the pressurize device can provide a power to move the material sample stored in the sample vessel 170, as to a plurality of sample vessels, a distribution device is provided to make all sample vessel connect to the gas source at one time, and in each pipe line, a on-off switch, such as valve, can be provided or not. And the distribution device can be any distributor known by the art; and the on-off switch can be any normal valve or special valve known by the art, such as, stopcock, electromagnetism valve, etc.

In another embodiment, referring to FIG. 12, each sample vessel 230 is connected to a pump 234 through a pipe 232, and the pump is connected to a connection element 236. The connection element 236 is connected to the materials-processing apparatus 240 through a pipe 238. In another embodiment, a power device is provided between the connection element and the processing apparatus, such that the sample vessel connected to the connection element is connected to the power device via the connection element.

Further, the inputting subsystem may comprise a flow measurement device to measure the quantity of the material sample entering the materials-processing apparatus. The flow measurement device can be a flow controller or a metric pump or any flow measurement device known by the art. There will be a plurality of connect manners between the sample vessel and the flow measurement device can connect with each other in various manners if only the flow measurement device can measure the quantity of the material sample entering the processing apparatus.

In another embodiment, referring to FIG. 13, a plurality of sample vessels 270 are connected to a connection element 274 through pipes respectively, and the connection element is connected to a flow controller 276, which is connected to a processing apparatus 280 through a pipe 278. In another embodiment, the flow measurement device is disposed to a pipe between the sample vessel and the connection element. In another embodiment, the connection element is in a movable manner as disclosed above, and the flow measurement device is disposed between the connection element and the processing apparatus, or between the sample vessel and the movable connection element.

Further, to simplify the materials-inputting sub system, the power device and the flow measurement device can be replaced by a metric pump, which has both functions of the two.

As to the way that the sample vessels are directly connected to the processing apparatus, there may be a fixed direct connection manner and a movable direct connection manner. Referring to FIG. 14, the fixed directly connection manner refers that a plurality of sample vessels 251, 252, 253 are directly connect to a processing apparatus 250. Referring to FIG. 15, the movable direct connection manner refers that a plurality of sample vessels 261, 262, 263, 264, 265 are movable (can be any movement manner known by the art, its movement track is not shown), and a connection between the outlet 266 of the sample vessel and an inlet 267 or 268 of the processing apparatus 250 is achieved through a movement of the sample vessel. There is no restriction as to the number of the inlet of the processing apparatus. Different sample vessels can respectively move to the corresponding inlets to complete the connection. A transportation manner of material from the sample vessel into the materials-processing apparatus can be any manner known by the art or the manners as disclosed above.

For the embodiments of the connection manner between the sample vessel and the processing apparatus as disclosed above, they can be used in combination. For example, a plurality of sample vessels is grouped into four groups, and the number of the sample vessels in each group can be different form each other or can be the same. For example, there are four groups of sample vessels respectively comprising 3 sample vessels, 4 sample vessels, 5 sample vessels, and 6 sample vessels. The sample vessels of the first group are immovably connect to a processing apparatus through a first connection element. The sample vessels of the second group are movably connected to the processing apparatus through a second connection element. The sample vessels of the third group are immovably and directly connected to the processing apparatus. The sample vessels of the fourth group directly and movably connected to the processing apparatus.

Further, the high throughput processing system with the materials-inputting subsystem in accordance with the present invention can further comprise an environmental temperature adjusting chamber, which can adjust the environmental temperature to increase fluidity of a sample with a relatively high viscidity. In one embodiment, referring to FIGS. 16 and 17, a temperature controlled chamber 20 comprises a body defining an inner surface 21 and an outer surface 22. There is a receiving room defined within the inner surface 21. An inner temperature control element 23 is provided on the inner surface 21 and an outer temperature control element 24 is provided on the out surface 22. Between the inner surface 21 and the outer surface 22, an insulator 25 is provided. The temperature change between the inner surface 21 and the outer surface 22 is minimized to reduce the rate of heat loss or gain to or from the inside of the chamber 20. The insulator may include one or more layers, and the insulator may be any insulating material, vacuum, or combination thereof. A multi-layer insulator may include multi-layers of insulating material or layers of insulating materials and vacuums in combination. For example, an insulator comprises three layers, wherein a top layer and a bottom layer are formed from different insulating materials, and the middle layer is formed by vacuum. There is no restriction as to the number of the layers of the insulator. Optionally, one or more reflective shields may be arranged between the inner surface 205 and the outer surface 215 of the chamber 200 to minimize heat loss through radiation.

Further, each part of the high throughput processing system may be equipped with a temperature adjusting device, so as to pertinently adjust the temperature of the each part. For example, each of the sample vessels and the connection elements, the processing apparatus, and the collecting subsystem may be coupled to a respective temperature adjusting device, such that the temperature of each part can be independently adjusted if needed. For example, in one embodiment, only the temperature of the sample vessel is adjusted. In another embodiment, both temperatures of the sample vessels and the processing apparatus are independently adjusted at same time. The temperature adjusting device can be any device with temperature adjusting function known by the art.

Further, the high throughput processing system with a materials-inputting subsystem in accordance with the present invention can be used in conjunction with automatization technology to achieve automatic operation of the system. The control manner of a control center controlling the whole system can be any control manner known by the art. For example, a control center comprises a computer system and corresponding input and output modules. Moreover, to increase the stability of the system, a programmable logic controller is provided to realize better bottom control. Any part of the system, such as, the processing apparatus, the temperature adjust device, flow control devices and pressure adjust devices, etc, may be contacted to the control center in order to report statuses of the system and receive instructions from the control center through any communication methods known by the art, including on-off signal or analog signal, such as, RS232, RS485 or 4-20 mA, etc.

A computer system (e.g., a server system) according to the present invention refers to a computer or a computer readable medium designed and configured to perform some or all of the methods as described in the present invention. A computer (e.g., a server) used herein may be any of a variety of types of general-purpose computers such as a personal computer, network server, workstation, or other computer platform now or later developed. As commonly known in the art, a computer typically contains some or all the following parts, for example, a processor, an operating system, a computer memory, an input device, and an output device. A computer may further contain other parts such as a cache memory, a data backup unit, and many other devices. It will be understood by those skilled in the relevant art that there are many possible configurations of the parts of a computer.

A processor used herein may include one or more microprocessor(s), field programmable logic arrays(s), or one or more application specific integrated circuit(s). Illustrative processors include, but are not limited to, Intel Corp's Pentium series processors, Sun Microsystems' SPARC processors, Motorola Corp.'s PowerPC processors, MIPS Technologies Inc.'s MIPs processors, Xilinx Inc.'s Vertex series of field programmable logic arrays, and other processors.

An operating system used herein comprises machine code that, once executed by a processor, coordinates and executes functions of other parts in a computer and facilitates a processor to execute the functions of various computer programs that may be written in a variety of programming languages. In addition to managing data flow among other parts in a computer, an operating system also provides scheduling, input-output control, file and data management, memory management, and communication control and related services, all in accordance with known techniques. Exemplary operating systems include, for example, a Windows operating system from the Microsoft Corporation, a UNIX or Linux-type operating system available from many vendors, another or a future operating system, and some combination thereof.

A computer memory used herein may be any of a variety of memory storage devices. Examples include any commonly available random access memory (RAM), magnetic medium such as a resident hard disk or tape, an optical medium such as a read and write compact disc, or other memory storage device. Memory storage device may be any of a variety of known or future devices, including a compact disk drive, a tape drive, a removable hard disk drive, or a diskette drive. Such types of memory storage device typically read from, and/or write to, a computer program storage medium such as, respectively, a compact disk, magnetic tape, removable hard disk, or floppy diskette. Any of these computer program storage media may be considered a computer program product. As will be appreciated, these computer program products typically store a computer software program and/or data. Computer software programs typically are stored in a system memory and/or a memory storage device.

As will be evident to those skilled in the relevant art, a computer software program of the present invention may be executed by being loaded into a system memory and/or a memory storage device through one of input devices. On the other hand, all or portions of the software program may also reside in a read-only memory or similar device of memory storage device, such devices not requiring that the software program first be loaded through input devices. It will be understood by those skilled in the relevant art that the software program or portions of it may be loaded by a processor in a known manner into a system memory or a cache memory or both, as advantageous for execution and used to perform a random sampling simulation.

In one embodiment of the invention, software is stored in a computer server that connects to an end user terminal, an input device or an output device through a data cable, a wireless connection, or a network system. As commonly known in the art, network systems comprise hardware and software to electronically communicate among computers or devices. Examples of network systems may include arrangement over any media including Internet, Ethernet 10/1000, IEEE 802.11x, IEEE 1394, xDSL, Bluetooth, LAN, WLAN, GSP, CDMA, 3Q PACS, or any other ANSI approved standard.

In another aspect, the present invention provides a high throughput system with a collecting subsystem. The high throughput system comprises an inputting subsystem, a processing apparatus connected to the material inputting subsystem and a collecting subsystem connected to the processing apparatus. The collecting subsystem comprises a plurality of collecting vessels for collecting materials from the processing apparatus. To some extend, the collecting subsystem can be regarded as a reversed the inputting subsystem, whose function is changed from providing samples into collecting samples. Thus detail about the collecting subsystem can refer to the disclosure for the inputting subsystem as disclosed above. An exemplary embodiment is provided as follows.

In an embodiment of the collecting subsystem, referring to FIG. 18, a high throughput processing system comprises an inputting subsystem (not shown), a processing apparatus 380 connected to the inputting subsystem and a collecting subsystem connected to the processing apparatus 380. The collecting subsystem comprises a connection element 360 and a plurality of collecting vessels 370, 371, 372, 373, 374, which are disposed on a base 378. One end of the connection element 360 is connected to an outlet of the processing apparatus 380 through a pipe 362, and the other end of the connection element 360 is movable, so as to connect with a selected collecting vessel (the movement track of the connection element 360 is figured by the broken line in figure, and the movement manner can be any movement manner known by the art, and can be different in different embodiments). The inputting subsystem can be any inputting subsystem known by the art or as disclosed by the present invention. The connect manners between the collecting vessel and the connection element, and setting of the base are similar to those disclosed in the embodiments of the materials inputting subsystem, only with sample flow directions reversed.

The processing apparatus used in the high throughput materials-processing system in accordance with the present invention, can be any type of the processing apparatus known by the art, such as, mixer, micromixer, reactor, microreactor, etc. The processing apparatus has applications including physical process of the materials samples and chemical process of the materials samples, and can be applied for mixing, extraction, synthesis, polymerization, emulsification, etc.

Further, for different embodiments of the microreactor, please refer to the disclosure of Zheng Yafeng, et al. “Research and Prospects of Microreactors” (article serial No.: TQ 03 A 1000-6613 (2004) 05-0461-07) chemical industry and engineering progress [J] 2004, 23(5). There are many kinds of microreactors, including integral reactor, reverse micellae microreactor, polymer microreactor, solid template microreactor, micro stripe reactor, and micro-polymerization reactor, etc. From an aspect of work model, there continuous style microreactor, semicontinuous style microreactor and intermission style microreactor. From an aspect of application, there are microreactors for plant use and microreactors for lab use, wherein the microreactor for lab use are generally used for medicaments screen, catalysts test, and process of development and optimization. And from the chemical reaction engineering aspect, the microreactor used has much to do with the reaction proceeded therein, and different types of the reactions require different types of the microreactors, so from the aspect of the reaction type, the microreactor can comprise gas-solid phase microreactor (embodiments can refer to Rebrov E V, de Croon M H J M, Schouten J C. [J]. Catal. Today, 2001, 69:183˜192; Srinivasan R, Hsing I M, Berger P E, et al. [J]. A ICh E J., 1997, 43:3059˜3069; Franz A, Jensen K F, Schmidt M A. Palladium Based Micromembranes for Hydrogen Separation and Hydrogenation/Dehydrogenation Reactions. In Ehrfeld W. Microreaction Technology: Industrial Prospects. Berlin: Springer, 2000, 267˜276), liquid-liquid phase microreactor (embodiments can refer to Wörz O Jäckel K P, Richter Th, et al. [J]. Chem. Eng. Sci., 2001, 56:1029˜1033; Wörz O Jäckel K P. [J]. Chem. Techn., 1997, 131 (26):130˜134; Floyd T M, Losey M W, Firebaugh S L, et al. Novel Liquid Phase Microreactors for Safe Production of Hazardous Specialty Chemicals. In: Ehrfeld W. Microreaction Technology: Industrial Prospects. Berlin: Springer, 2000, 171˜180; Daykin R N C, Haswell S J. [J]. Anal., Chim. Acta., 1995, 313 (3):155˜159), gas-liquid phase microreactor (embodiments can refer to Haverkamp V, Emig G Hessel V, et al. Characterization of a Gas/Liquid Microreactor, the Microbubble Column: Determination of Specific Interfacial Area[C]. Proc. of the 5th Int. Conf. on Microreaction Technology, IMERT 4, Strasbourg, France, 2001; Losey M W, Schmidt M A, Jensen K F. A Micro Packed-bed Reactor for Chemical Synthesis. In: Ehrfeld W. Microreaction Technology: Industrial Prospects. Berlin: Springer, 2000, 277˜286), gas-liquid-solid phase microreactor (embodiments can refer to Losey M W, Schmidt M A, Jensen K F. [J]. Microengineering, 2000, 6:285˜289; Jähnisch K, Baerns M, Hessel V, et al. [J]. Fluorine Chem., 2000, 105 (1):117˜128), electrochemical microreactor for electrochemistry reaction and photochemical microreactor for photochemical reaction (embodiments can refer to Löwe H, Ehrfeld W, Küpper M, et al. Electrochemical Microreator: A New Approach in Microreaction Technology [C]. 3rd Int. Conf. on Microreaction Technology, Proc. of IMERT 3, Berlin, 2000; Lu H, Schmidt M A, Jensen K F. Photochemical Reactions and on-line Monitoring in Microfabricated Reactors [C]. Proc. of the 5th Int. Conf. on Microreaction Technology, IMERT 4, Strasbourg, France, 2001; Lu H, Schmidt M A, Jensen K F. [J]. Lab on a Chip., 2001, 1:22˜28). The detail description of the microreactors disclosed above can refer to the disclosure of the cited articles, which are incorporated here by reference.

For different embodiments of the micromixer, please refer to Zhu Li, et al., “Research and Prospects of Micromixes” (article serial No.: 0353.5 A 1671-4776 (2005) 04-0164-08) Micronanoelectronic Technology [J] 2005, 4. There are an initiative micromixer and a passivity micromixer. The initiative micromixer can comprise an ultrasonic micromixer for microfluidic system reported by Zhen YANG et al. (embodiments can refer to YANG Z, MATSUMOTO S, GOTO H, et al. Ultrasonic micromixer for microfluidic system [J]. Sensors and Actuators A, 2001, 93:266-272), an actively controlled micromixer reported by Frédéric Bottausci et al. (embodiments can refer to RIC BOTTAUSCI F, CARDONNE C, MEZIĆ I, et al. An actively controlled micromixer: 3-D aspect [EB/OL]. http://www.engineering. ucsb.edu/˜mgroup), an electroosmosis micromixer reported by Peter Huang et al. (embodiments can refer to HUANG P, BREUER K S. Performance and scaling of an mixer [EB/OL]. http://microfluidics.engin.brown.edu/ breuer_paper/Conferences), two kinds of micro-apparatus for mixing fluids and particulate reported by Yi-Kuen Lee et al. (embodiments can refer to LEE Y K, DEVAL J, TABELING P, et al. Chaotic mixing in electrokinetically and pressure driven micro flows [A]. The 14th IEEE Workshop on MEMS Interlaken [C]. Jan: Switzerland, 2001.), a minute magneto hydro dynamic (MHD) mixer reported by Bau et al. (embodiments can refer to BAU H H, ZHONG J H, YI M Q. A minute magneto hydro dynamic (MHD) mixer [J]. Sensors and Actuators B, 2001, 79:207-215), a continuous micromixer with pulsatile micropumps reported by Deshmukh et al. (embodiments can refer to DESHMUKH A A, LIEPMANN D, PISANO A. P Continuous micromixer with pulsatile micropumps [EB/OL]. http://www.me.berkeley.edu/˜liepmann/assets). The passivity micromixer comprises a T-shaped micromixer reported by Seck Hoe Wong et al. (embodiments can refer to WONG S H, WARD M C L, WHARTON C W. Micro T-mixer as a rapid mixing micro mixer [J]. Sensors and Actuators B, 2004, 100:359-379), a rapid vortex micromixer reported by S. Böhm et al. (embodiments can refer to BÖHM S, GREINER K, SCHLAUTMANN S, et al. A rapid vortex micromixer for studying high-speed chemical reactions [EB/OL]. http//www.coventor. com/media/ papers.), a cross fluid joint micromixer reported by Xu Yi et al. (embodiments can refer to XU YI BESSOTH F, MANZ A. “STUDY ON DESIGNING AND PERFORMANCE OF MICRO CHIP COMPRISING MICROMIXER” [J]. JOURNAL OF INSTRUMENTAL ANALYSIS, 2000, 19 (4):39-42), a micromixer reported by Dertinger et al. (embodiments can refer to DERTINGER S K W, CHIU D T, JEON N L, et al. Generation of gradients having complex shapes using microfluidic), a chaotic mixer reported by Stroock et al. (embodiments can refer to STROOCK A D, DERTINGER S K W, AJDARI A, et al. Chaotic mixer for microchannels [J]. Science, 2002, 295:647-651), a membrane dispersion micromixer reported by Luo Guangsheng et al.( embodiments can refer to: Luo Guangsheng, Chen Guaiguang, Xu Jianhong et al. “micromixer and its performance research progress” [J], Modern Chemical Industry 2003, 23(8): 10-13). The detail description of the micromixers disclosed above can refer to the disclosure of the cited articles, which are incorporated here by reference.

Further, in one embodiment, a processing apparatus, which can refer to the disclosures of U.S. Pat. Nos. 5,538,191, 6,471,392 and 6,742774, comprises a work part and a driving part. The work part comprises a stator and a rotor in the stator. A processing chamber is formed between the stator and the rotor. The rotor is driven by the driving part. More details of the processing apparatus are described in U.S. Pat. Nos. 5,538,191, 6,471,392 and 6,742774, which are incorporated here by reference.

In another embodiment, a processing apparatus, which can refer to the disclosure of a PCT application: PCT/CN2005/002177 filed in Dec. 13, 2005 by the applicants of the present invention, comprises a work part and a driving part. The work part comprises a first element and a second element disposed in the first element. The first element and the second element form therebetween a chamber for receiving samples. The second element can be driven to rotate relate to the first element by the driving part. A surface of the first element or second element facing to the chamber is unsmooth. More details of the processing apparatus are described in the PCT patent application PCT/CN2005/002177, which is incorporated here by reference.

In another aspect, the present invention provides a processing system, which comprises an inputting subsystem in accordance with the present invention, a collecting subsystem in accordance with the present invention and a processing apparatus. Detail description of the inputting subsystem in accordance with the present invention and the collecting subsystem in accordance with the present invention can refer to the above disclosure.

In another aspect, the present invention provides a high throughput inputting method for continuously inputting batches of samples into a processing system, comprising the steps of, firstly, providing a plurality of sample vessels and grouping these sample vessels into a plurality of groups each group comprising at least one material different from the others; secondly, sequentially connecting the groups of sample vessels to a materials processing apparatus, so as to transport the materials of each group into the materials processing apparatus.

Further, as to the high throughput inputting method in accordance with the present invention, the numbers of the samples vessels in different group can be same or not, and each sample vessel can be used by different groups or used by only one certain group, based on different request. Specially, if there are four sample vessels provided, and given that each sample vessel can be used by different groups, there are eleven groups, calculated by “permutation and combination” method. These eleven groups include six groups each containing two sample vessels; four groups each containing three sample vessels and one group containing four sample vessels. If each sample vessel is used by only one group, the number of groups relatively decreases. Since the variation of the embodiments can be understood by those skilled in the art, no more explanations are provided

Further, as the connection manner between the sample vessels and the materials processing apparatus and the transportation manner of the materials into the materials processing apparatus can be the embodiments disclosed by the present invention or known by the art.

In another aspect, the present invention provides a high throughput collecting method, comprises the steps of, providing a plurality of collecting vessels, connecting each collecting vessel to a material processing apparatus one by one to realize a continuously materials collection. The connection manner between the collecting vessels and the material processing apparatus and the material transportation manner from the material processing apparatus into the collecting vessel can refer to the disclosure of the embodiments disclosed above or the corresponding manners known by the art.

In another aspect, the present invention provides a clean method for cleaning the high throughput material processing system with an inputting subsystem in accordance with the present invention, which comprises steps of, selecting at least one of the sample vessels of the multi material inputting subsystem to store a cleaning material for cleaning the high throughput material processing system; connecting the selected sample vessel stored with cleaning material to the material processing apparatus to transport the cleaning material therein to the material processing apparatus when needed; and then expelling the used cleaning material from the material processing apparatus to complete the cleaning of the system.

Further, the cleaning material used in the clean method in accordance with the present invention, can be in liquid or gas state. Specially, liquid state cleaning materials includes water, ethanol, organic solvent and other liquid state cleaning materials known by the art. Gas state cleaning materials include a compress air, nitrogen and helium, etc. Different kinds of cleaning materials are selected for different kinds of material samples. Further, duration time for inputting the cleaning material can be adjusted; and it can be several seconds, tens of seconds, several minutes, and even several hours, specially, 4 seconds, 6 seconds, 8 seconds, 10 seconds, 15 seconds, 20 seconds, 30 seconds, 40 seconds, 10 minutes, 30 minutes, 2 hours, 3 hours, etc.

Further, in an embodiment of the cleaning method in accordance with the present invention, there are two sample vessels selected for storing cleaning materials. One is for storing gas state cleaning material, and the other is for storing liquid state cleaning material. So when needed, the liquid state cleaning material or gas state cleaning material or both can be selected to be inputted into the system. When the both of them are selected to be inputted, a transportation manner thereof depends on different requests. For example, it can be firstly inputting gas state cleaning material for several minutes, then inputting liquid state cleaning material for several minutes; or circularly inputting liquid state material and gas state material, each for tens of seconds.

As to the cleaning method in accordance with the present invention, the inputting manner of the cleaning materials is same as the inputting manner of the samples, and its embodiments can refer to the above corresponding disclosure. However, it should be noticed that a periphery of the docking port of the connection element of the movable connection manner may be contaminated by sample and should be cleaned for better clean effect.

In an embodiment, referring to FIG. 19, a movable first connection element 450 (different embodiments can refer to the above corresponding disclosure of the present invention) directly move into a sample vessel stored with a cleaning material. The cleaning material (not shown) enters the first connection element through a docking port 452 along an arrow direction as shown in the FIG. 19, and cleans the periphery of the docking port 452 at the same time. Then the cleaning material enters a processing apparatus 460 through a pipe 456 connected therebetween to begin cleaning the processing apparatus. Finally, the used cleaning material is expelled out of the processing apparatus through a docking port 472 of a second movable connection element, which is connected to the processing apparatus through a pipe 476 connected there between, and thus cleaning of the system is finished. At the same time, the docking port of the second movable connection element had better move to a position rightly above a bucket 480 for collecting used materials, so as to collect the used cleaning material. A shape of the bucket 480 is preferably designed the same or familiar as shown in the figures, so as to clean the periphery 473 of the docking port 472 of the second movable connection element 470 during expelling of the used cleaning material. In another embodiment, the used cleaning material can be expelled through an outlet of the materials-processing apparatus and collected by the collecting subsystem. In another embodiment, the cleaning material can be inputted through the fixed connection element, detail description for which can refer to the above corresponding disclosure. So with the cleaning method in accordance with the present invention, the processing system can continuously proceeding the material sample processing and the system cleaning without interruption, just like continuously proceeding batches of sample processing with some batches of samples are replaced by the cleaning material. Thus an efficiency of the system is increased.

Compare to the prior art, the inputting subsystem of the processing system with 16 sample vessels for example, in accordance with the present invention, can continuously input 120 groups each comprising two samples, or 560 groups each comprising three samples into the processing apparatus, if proper a connection manner as disclosed in the present invention is chosen (there will be more embodiments for the groups of the sample vessels based on the permutation and combination theory). Since there is no restriction as to the number of the sample vessels, there is no restriction as to the number of the groups for inputting, that means the operator can decide the number of the sample vessels. Therefore, the continuous inputting can increase the efficiency of the system in one hand and decrease the risk of mistakes of manual operation in the other hand, and the high throughput inputting brings high throughput material processing of the system. Moreover, with the combination of the collecting subsystem in accordance with the present invention, the samples processed can be collected orderly and continuously, so as to further increase the efficiency of the system and overcome the deficit of the artificially interval samples collecting of the prior art. Finally, with the combination of the cleaning method in accordance with the present invention, the cleaning of the system can proceed next to a sample process with no interruption, so as to overcome the deficits of the prior art in cleaning aspect and further increase the efficiency of the system.

### BRIEF DESCRIPTION OF THE DRAWINGS

FIG. 1 is a scheme of an embodiment of a fixed connection between a plurality of sample vessels of an inputting subsystem in accordance with the present invention and a processing apparatus through a connection element;

FIG. 2 is a scheme of another embodiment of the fixed connection between a plurality of sample vessels of the inputting subsystem in accordance with the present invention and the processing apparatus through the connection element;

FIG. 3 is a scheme of yet another embodiment of the fixed connection between a plurality of sample vessels of the inputting subsystem in accordance with the present invention and the processing apparatus through the connection element;

FIG. 4 is a scheme of an embodiment of a movable connection between a plurality of sample vessels of the inputting subsystem in accordance with the present invention and the processing apparatus through the connection element;

FIG. 5 is a scheme of another embodiment of the movable connection between a plurality of sample vessels of the inputting subsystem in accordance with the present invention and the processing apparatus through the connection element;

FIG. 6 is a scheme of an embodiment of the fixed connection manner and the movable connection in combination between a plurality of sample vessels of the inputting subsystem in accordance with the present invention and the processing apparatus through the connection elements;

FIG. 7 is a scheme of yet another embodiment of the movable connection between a plurality of sample vessels of the inputting subsystem in accordance with the present invention and the processing apparatus through the connection element, wherein the sample vessels are disposed on a base;

FIG. 8 is a scheme of an embodiment of a plurality of sample vessels disposed on the base;

FIG. 9 is a scheme of an embodiment of a sample vessel in accordance with the present invention;

FIG. 10 is a scheme of an embodiment of the sample vessel connecting to the connection element;

FIG. 11 is a scheme of an embodiment of the inputting subsystem in accordance with the present invention equipped with a power device;

FIG. 12 is a scheme of another embodiment of the inputting subsystem in accordance with the present invention equipped with a plurality of power devices;

FIG. 13 is a scheme of an embodiment of a plurality of sample vessels of the inputting subsystem in accordance with the present invention directly connecting to the processing apparatus;

FIG. 14 is a scheme of an embodiment of a plurality of sample vessels movably connecting to the processing apparatus;

FIG. 15 is a scheme of an embodiment of cleaning the movable connection element;

FIG. 16 is a scheme of an embodiment of a temperature adjusting chamber in accordance with the present invention;

FIG. 17 is a cross-section view of the FIG. 16 along A-A direction;

FIG. 18 is a scheme of an embodiment of a material collecting subsystem in accordance with the present invention;

FIG. 19 is a scheme of an embodiment of the cleaning method in accordance with the present invention;

FIG. 20 is a scheme of an embodiment of a high throughput processing system in accordance with the present invention.

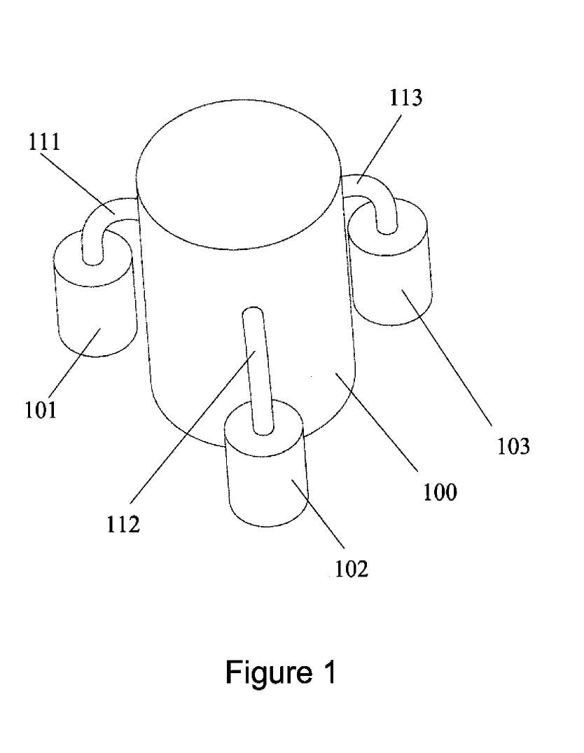

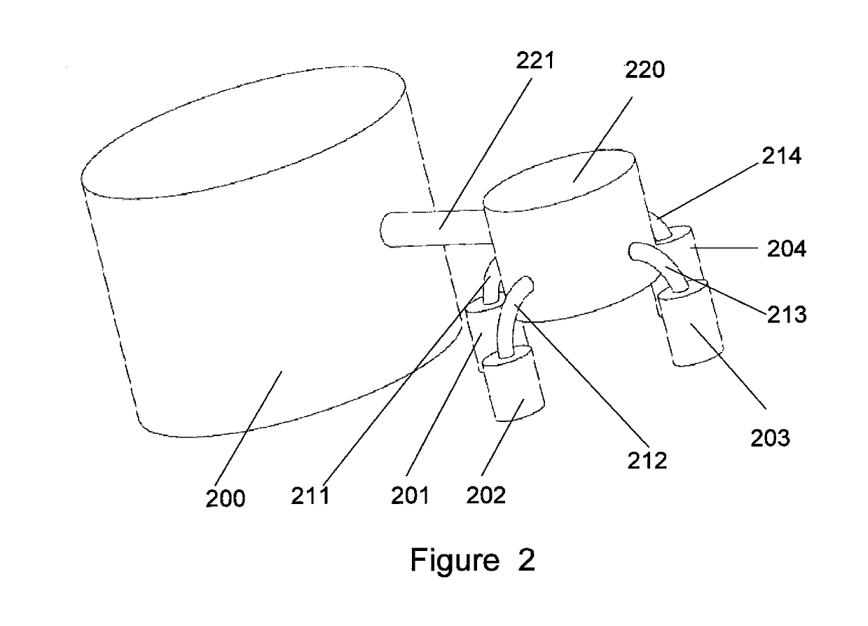

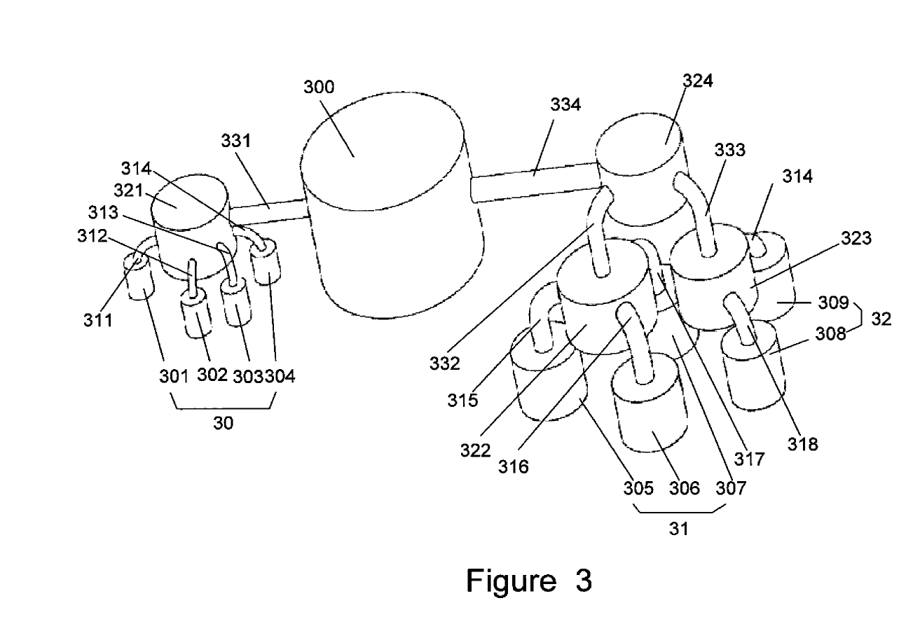

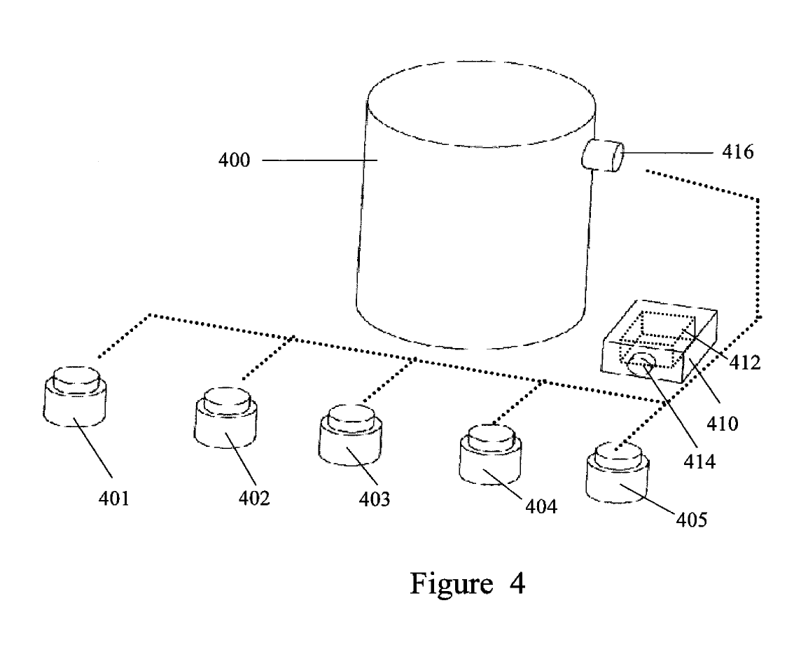

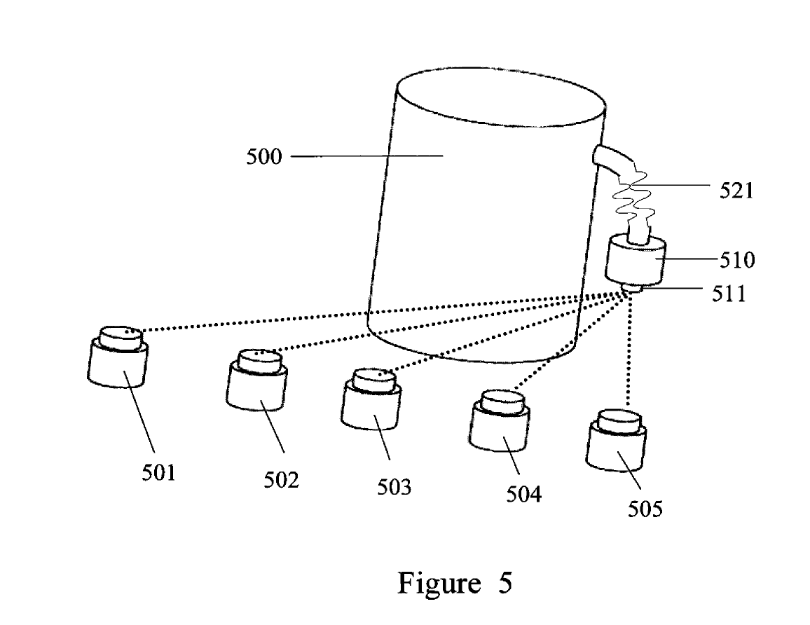

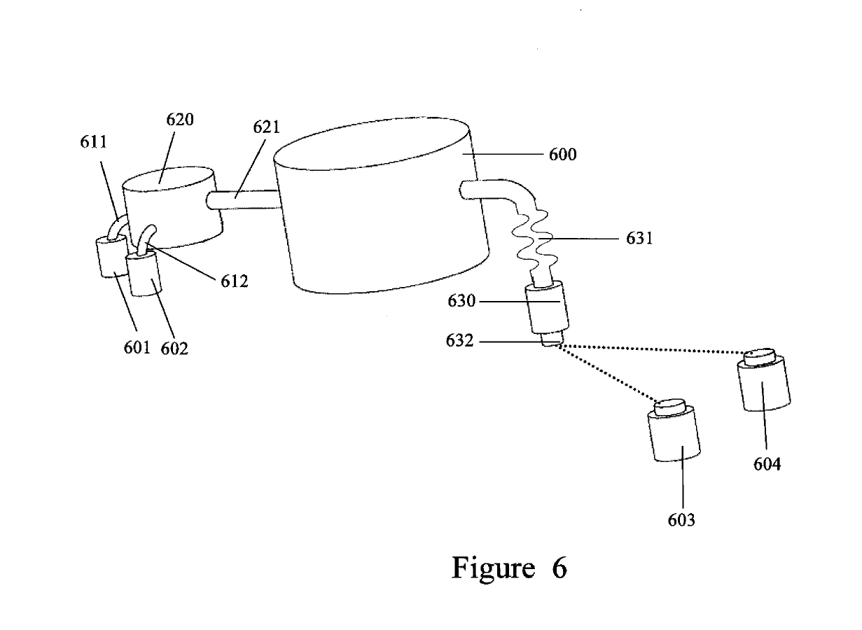

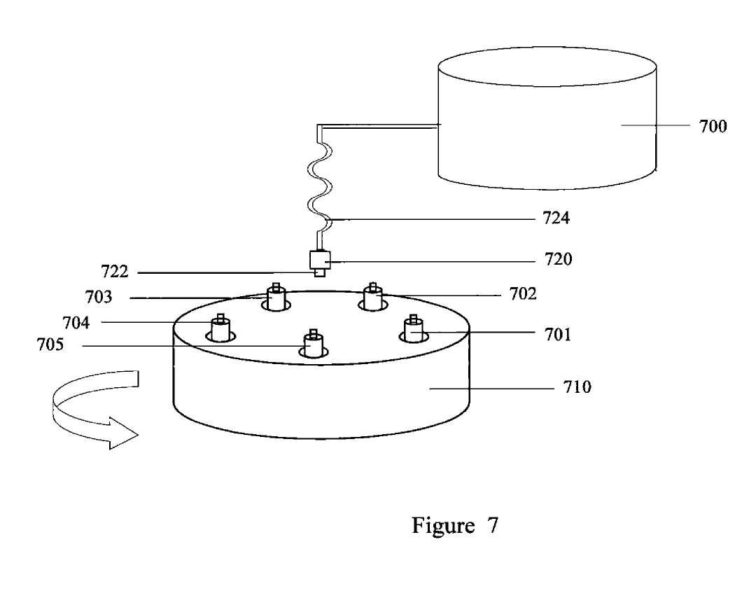

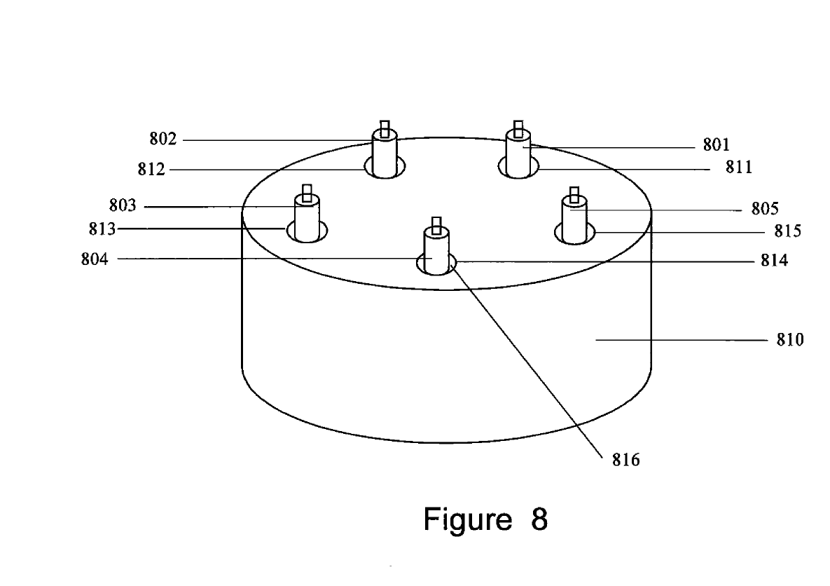

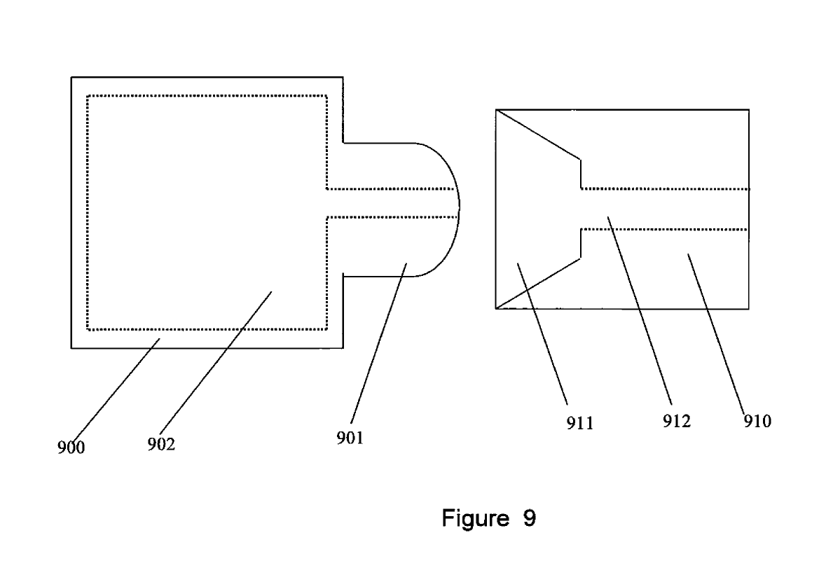

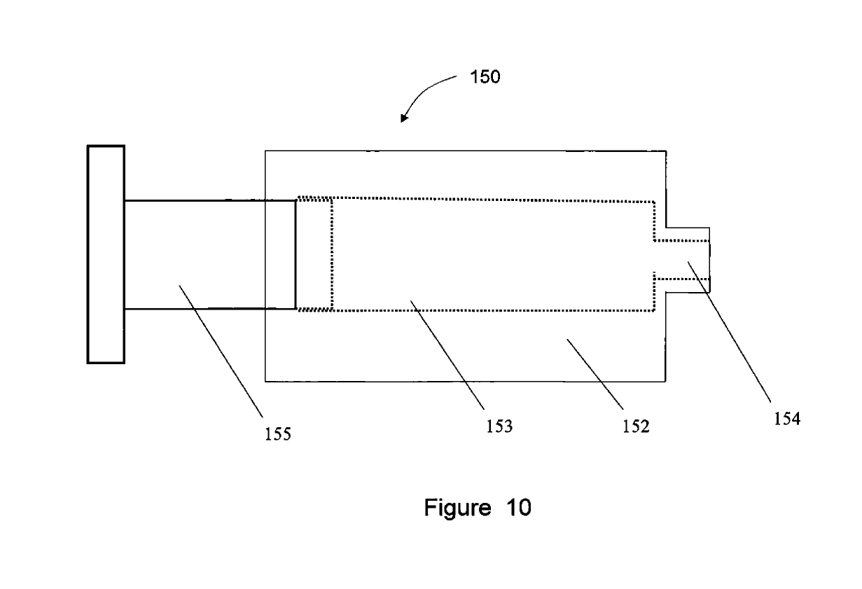

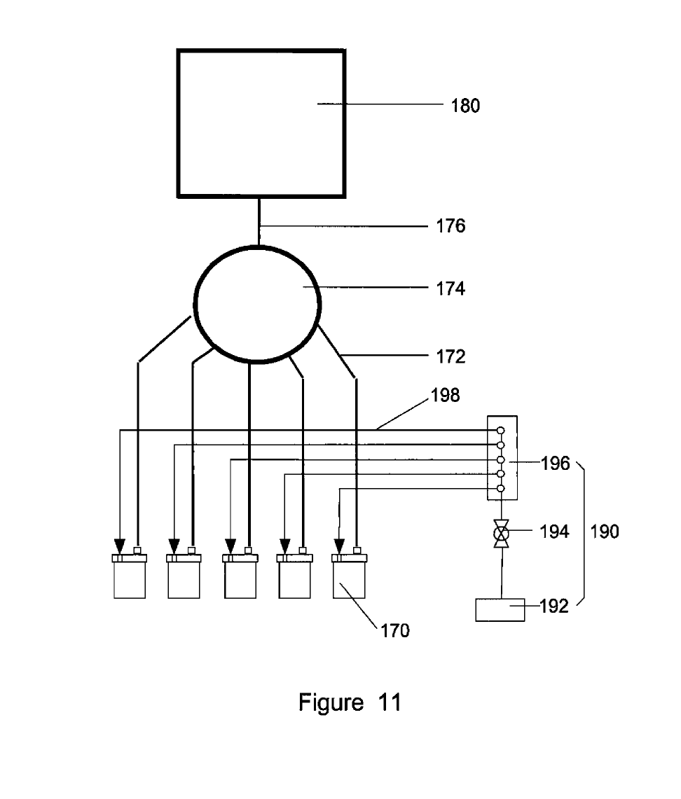

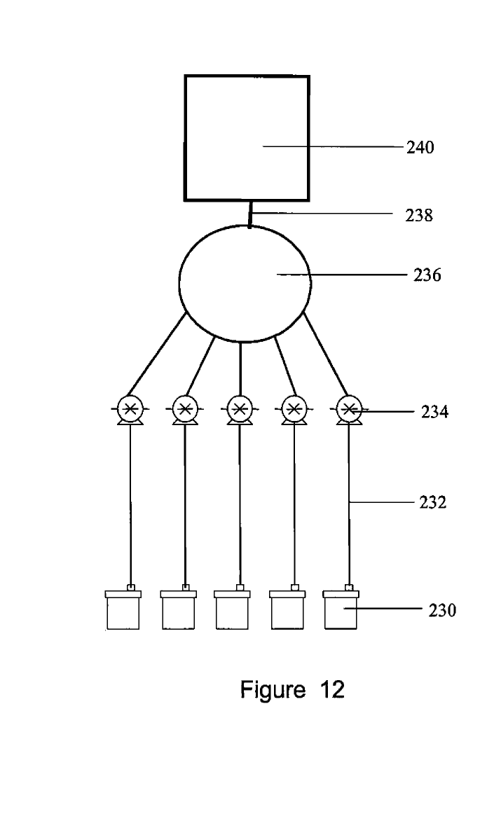

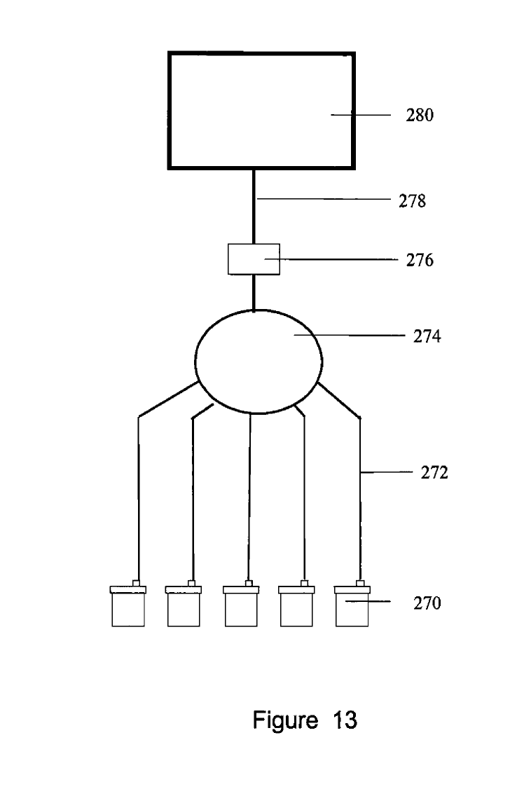

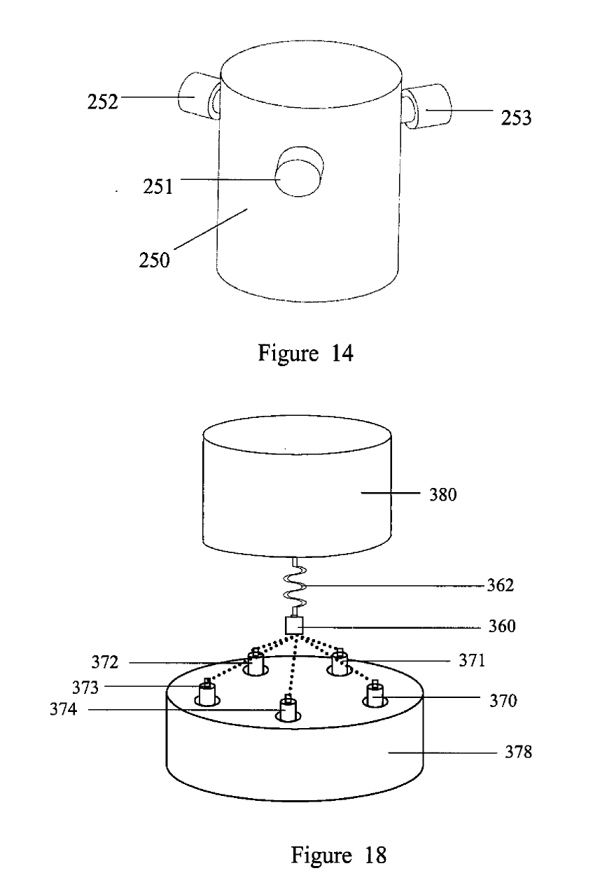

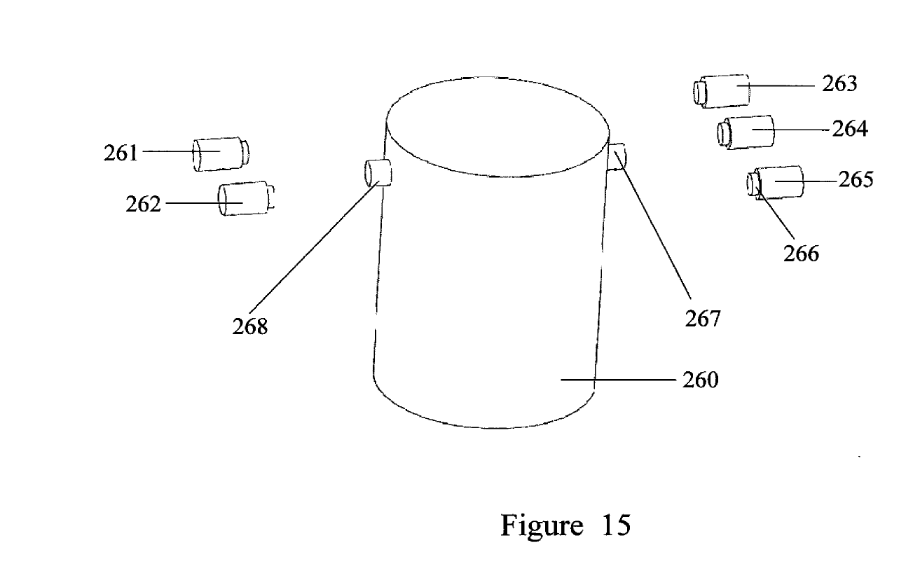

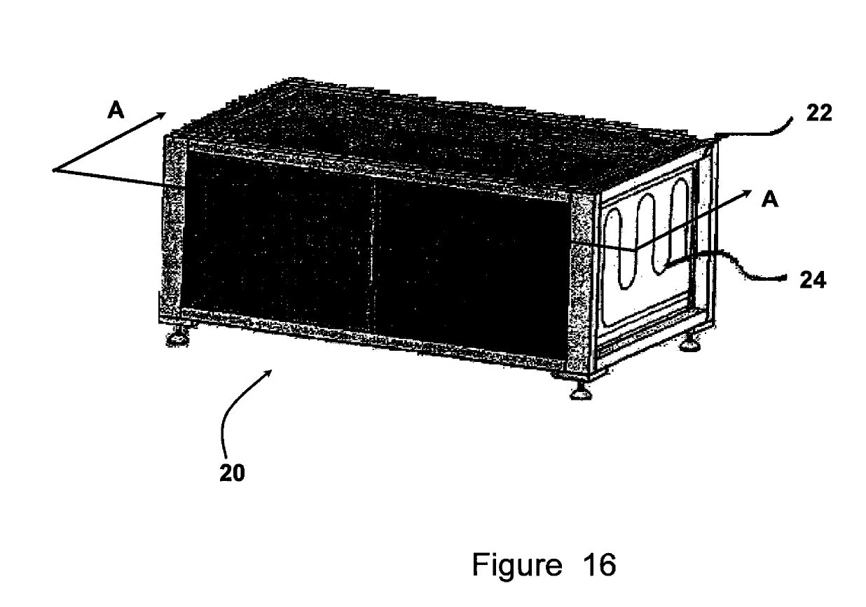

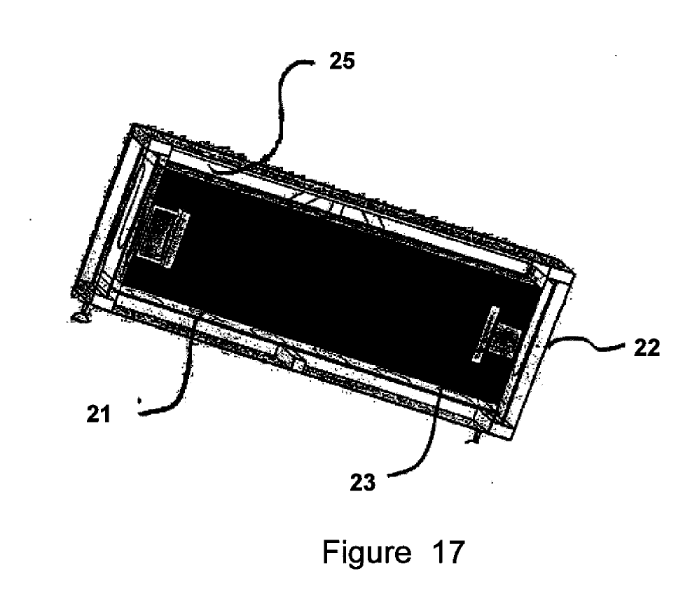

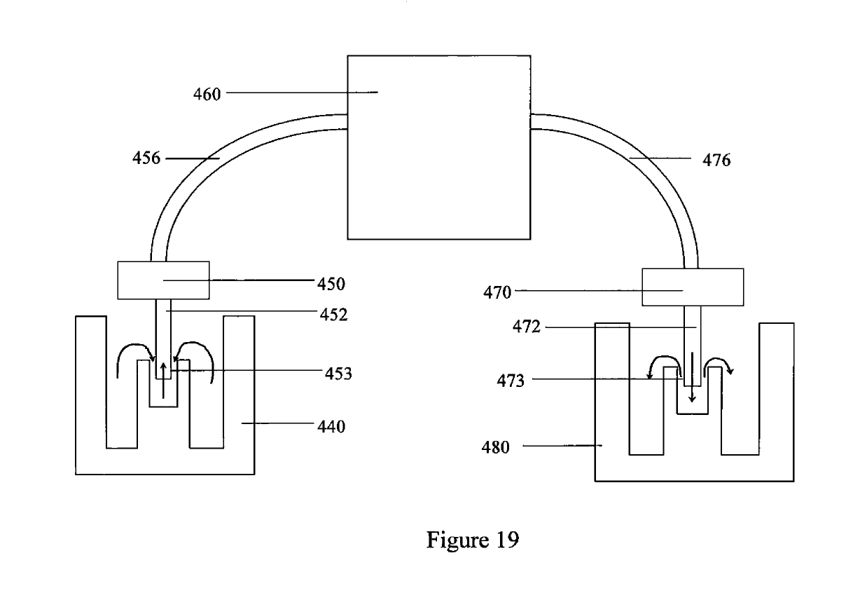

### DETAIL DESCRIPTION OF THE EMBODIMENTS

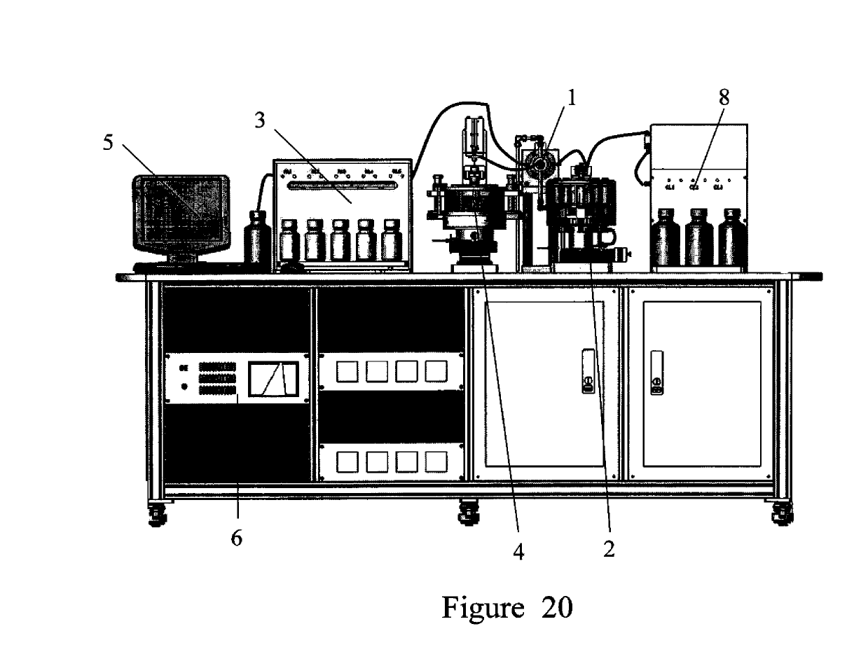

Please refer to FIG. 20, the high throughput system in accordance with the present invention, comprises an inputting subsystem, a processing apparatus and a collecting subsystem. The inputting subsystem comprises first and second inputting modules 2 and 3 wherein the first inputting module 2 is connected to the processing apparatus 1 through a first connection element in a fixed connection manner (the detail of the connection manner are not shown) and has two sample vessels stored with respective liquid cleaning material and gas state cleaning materials therein. The second inputting module comprises a plurality of sample vessels connected to the processing apparatus through a second connection element in a movable connection manner (the detail of the connection manner are not shown), and an independent cleaning component 8 providing a plurality of cleaning materials therein. The collecting subsystem is connected to the processing apparatus through a third connection element in the movable connection manner. The high throughput processing system further comprises a control center 5 to automate the system, comprising a computer system and the corresponding input and output electrical control modules.

The operation of the system comprises the following steps:

1. Electric initialization: launching the software to perform an operation of the electrical initialization;

2. Preparation of the experiment: loading the samples and cleaning materials into the corresponding sample vessels of the inputting subsystem, specially, loading the samples and the cleaning materials for the first and second inputting modules into their respective sample vessels;

3. Preparation of the processing: checking each parts of the system to make sure the system is ready for sample processing;

4. Samples processing: selecting one sample vessel from each inputting module to connect to the processing apparatus, and then transporting the samples stored therein into the processing apparatus to begin processing the samples;

5. Product collection: connecting one of the collection vessels to the materials-processing apparatus to collect products of the sample process, when the process is finished;

6. Cleaning: connecting the sample vessels stored with the cleaning materials in each inputting module to the processing apparatus to transport the cleaning materials stored therein into the processing apparatus to begin a cleaning process;

7. Proceeding process of the samples of the next group: repeating the fourth step

8. Independent cleaning of the system: cleaning all the sample vessels and collecting vessels of the system for next usage

9. Shut down the system.

Taking mixing of crude oil and ionic liquids as an example, ionic liquids are promising with an application to extract some materials from the crude oil. However, there are thousands of types of ionic liquids, so it is very difficult to quickly find a proper ionic liquid. The high throughput processing system in accordance with the present invention is very suitable for this kind of job.

During the process, the crude oil and the ionic liquid samples are loaded into the respective sample vessels respectively in the first and second inputting modules. During experiment, some of the ionic liquid samples with a high viscidity are heated to decrease their viscidity. At the same time, to simulate real conditions of the industrial application, environment conditions of the samples, including temperature, pressure, etc. should be adjusted as similar to the real conditions of industrial application. Therefore, sometimes, the crude oil and ionic liquid samples can be heated to some extent. Therefore the inputting subsystem can be equipped with temperature adjusting function. The temperature may be adjusted in a range from room temperature to 65° C.

During the process of the cleaning, since there are too many kinds of ionic liquids, three cleaning channels are set for an ionic liquid inputting system. The number of the cleaning channels for the ionic liquid inputting system can expand according to specific request. Relatively, the crude oil has a less number of types, and different types of crude oil are similar in property, so only one cleaning channel is set for a crude oil inputting system. The number of the cleaning channels for the crude oil inputting system can also expand according to specific request. The liquid state cleaning material can be pressurized by nitrogen to prevent the metric pump from being invalid.

The system in accordance with the present invention can perform experiments for mixing 10 types of the ionic liquids with 5 types of crud oil in a day, that is to say, 50 kinds of products can be obtained in a day, so the efficiency of the system is comparable high.

1-83. (canceled)

84. A materials-processing system, comprising
a first plurality of sample vessels each having an outlet;
a processing apparatus having a stator, a rotor, a chamber formed between a rotor and a stator, a first input, and an output; and
a first connection device to selectively connect the outlets of some or all of the sample vessels to the first input of the processing apparatus.

85. The materials-processing system according to claim 84, wherein the first connection device is configured to connect to two or more outlets of the sample vessels directly.

86. The materials-processing system according to claim 84, wherein the first connection device includes a selection valve.

87. The materials-processing system according to claim 84, wherein the first connection device is movable such that selective connection of the sample vessels to the first input is accomplished by moving the first connection device.

88. The materials-processing system according to claim 84, wherein the sample vessels are movable such that selective connection of the sample vessels to the processing apparatus is accomplished by moving some or all of the sample vessels.

89. The materials-processing system according to claim 84, wherein the materials-processing system further comprises a second connection device and a second plurality of sample vessels, and the processing apparatus includes a second input; and the second connection device is used to selectively connect the outlets of some or all of the second plurality of sample vessels to the second input.

90. The materials-processing system according to claim 89, wherein the second connection device is movable such that selective connection of the second plurality of sample vessels to the second input is accomplished by moving the second connection device.

91. The materials-processing system according to claim 84, wherein the materials-processing system further comprises a plurality of sample collecting vessels each having an inlet, and a third connection device; and the third connection device is used to selectively connect the inlet of at least one of the collecting vessels to the output of the processing apparatus.

92. The materials-processing system according to claim 81, wherein the third connection device connects to two or more inlets of the sample collecting vessels directly.

93. The materials-processing system according to claim 91, wherein the third connection device includes a selection valve.

94. The materials-processing system according to claim 91, wherein the third connection device is movable such that selective connection of the sample collecting vessels to the output is accomplished by moving the third connection device.

95. The materials-processing system according to claim 91, wherein the sample collecting vessel are movable such that selective connection of the sample collecting vessels to the processing apparatus is accomplished by moving some or all of the sample collecting vessels.

96. A method for processing materials, comprising
selectively connecting at least one of a first plurality of three or more sample vessels to a processing apparatus having a stator, a rotor, a chamber formed between a rotor and a stator, a first input, and an output;
transporting materials stored in the at least two of the first plurality of three or more sample vessels into the processing apparatus through the first input;
processing the materials in the processing apparatus by causing at least one of the stator and rotor to rotate relative to each other; and
outputting processed materials into one or more collecting vessels through the output.

97. The method of claim 96, wherein selectively connecting includes adjusting a selection valve coupled between the processing apparatus and the first plurality of sample vessels.

98. The method of claim 96, wherein selectively connecting includes moving a first selection device coupled between the processing apparatus and the first plurality of sample vessels.

99. The method of claim 96, wherein selectively connecting includes moving some or all of the first plurality of sample vessels relative to the processing apparatus.

100. The method of claim 96, wherein the processing apparatus further includes a second input, and the method further comprises selectively connecting at least one of a second plurality of sample vessels to the processing apparatus and transporting materials from the at least one of the second plurality of sample vessels into the processing apparatus through the second input.

101. The method of claim 100, wherein selectively connecting includes moving a second connection device coupled between the processing apparatus and the second plurality of sample vessels.

102. The method of claim 96, further comprising selectively connecting at least one of a plurality of collecting vessels to the output of the processing apparatus.

103. The method of claim 102, wherein selectively connecting at least one of a plurality of collecting vessels to the output of the processing apparatus includes adjusting a selection valve coupled between the processing apparatus and the plurality of collecting vessels.
